(19) United States
(12) Patent Application Publication
(10) Pub. No.: US 2025/0222031 A1
(43) Pub. Date: Jul. 10, 2025

(54) Treatment of Disc Degeneration using B Regulatory Cells

(71) Applicant: CREATIVE MEDICAL TECHNOLOGIES, INC., Phoenix, AZ (US)

(72) Inventors: Thomas Ichim, San Diego, CA (US); Timothy Warbington, Phoenix, AZ (US)

(73) Assignee: CREATIVE MEDICAL TECHNOLOGIES, INC., Phoenix, AZ (US)

(21) Appl. No.: 19/015,315

(22) Filed: Jan. 9, 2025

Related U.S. Application Data
(60) Provisional application No. 63/619,230, filed on Jan. 9, 2024.

(51) Int. Cl. A61K 35/17; A61K 45/06

(52) U.S. Cl. CPC A61K 35/17; A61K 45/06; C12N 2501/998; C12N 2501/999; C12N 2502/1352

(57) ABSTRACT

Compositions of matter, interventions and protocols for treatment of degenerative disc disease through administration of B regulatory cells and/or stimulation of B regulatory cell generation. B regulatory cells can be administered intradiscally in order to support regenerative processes of endogenous nucleus pulposus cells or administered regenerative cells. Peri-spinal administration of B regulatory cells can alter the microenvironment to support activity of therapeutic cells capable of increasing extracellular matrix quality and quantity. B regulatory cells can be expanded in vitro from progenitor cells including induced pluripotent stem cells, or may be utilized from adult progenitor sources such as peripheral blood, cord blood, and bone marrow.

### CROSS-REFERENCE TO RELATED APPLICATIONS

The present application claims benefit of U.S. Provisional Patent Application Ser. No. 63/619,230, filed on Jan. 9, 2024 entitled TREATMENT OF DISC DEGENERATION USING B REGULATORY CELLS, the contents of which are incorporated herein by reference in its entirety.

### FIELD OF THE INVENTION

The invention relates to the field of regenerative medicine, more specifically, the invention pertains to the field of inducing regenerative processes to intervertebral disc that are undergoing loss of function, more specifically, the invention relates to treatment of disc degenerative disease using B regulatory cells.

### BACKGROUND OF THE INVENTION

The intervertebral discs make up one fourth of the spinal column's length. There are no discs between the Atlas (C1), Axis (C2), and Coccyx. Discs are not vascular and therefore depend on the end plates to diffuse needed nutrients. The cartilaginous layers of the end plates anchor the discs in place.

The intervertebral discs are fibrocartilaginous cushions serving as the spine's shock absorbing system, which protect the vertebrae, brain, and other structures (i.e. nerves). The discs allow some vertebral motion: extension and flexion. Individual disc movement is very limited-however considerable motion is possible when several discs combine forces.

Intervertebral discs are composed of an annulus fibrosus and a nucleus pulposus. The annulus fibrosus is a strong radial tire-like structure made up of lamellae; concentric sheets of collagen fibers connected to the vertebral end plates. The sheets are orientated at various angles. The annulus fibrosus encloses the nucleus pulposus.

Although both the annulus fibrosus and nucleus pulposus are composed of water, collagen, and proteoglycans (PGs), the amount of fluid (water and PGs) is greatest in the nucleus pulposus. PG molecules are important because they attract and retain water. The nucleus pulposus contains a hydrated gel-like matter that resists compression. The amount of water in the nucleus varies throughout the day depending on activity. As people age, the nucleus pulposus begins to dehydrate, which limits its ability to absorb shock. The annulus fibrosus gets weaker with age and begins to tear. While this may not cause pain in some people, in others one or both of these may cause chronic pain.

Pain due to the inability of the dehydrating nucleus pulposus to absorb shock is called axial pain or disc space pain. One generally refers to the gradual dehydration of the nucleus pulposus as degenerative disc disease. When the annulus fibrosus tears due to an injury or the aging process, the nucleus pulposus can begin to extrude through the tear. This is called disc herniation. Near the posterior side of each disc, all along the spine, major spinal nerves extend out to different organs, tissues, extremities etc. It is very common for the herniated disc to press against these nerves (pinched nerve) causing radiating pain, numbness, tingling, and diminished strength and/or range of motion. In addition, the contact of the inner nuclear gel, which contains inflammatory proteins, with a nerve can also cause significant pain. Nerve-related pain is called radicular pain.

Herniated discs go by many names and these can mean different things to different medical professionals. A slipped disc, ruptured disc, or a bulging disc can all refer to the same medical condition. Protrusions of the disc into the adjacent vertebra are known as Schmorl's nodes.

The treatment of degenerative disc disease and associated spine ailments is one of the most-costly medical conditions with an estimated annual direct cost in the United States of 33 billion and total annual societal cost exceeding 100 billion dollars. Indeed, in one's lifetime most individuals will experience an episode of significant back and or neck pain. Although most individuals will improve with non-operative intervention, a significant percentage will go on to require costly surgery or other medical intervention.

The etiology of chronic back pain is multi-factorial, but in a significant proportion of patients, degenerative disc disease is the underlying cause. As an individual ages, the intervertebral disc looses water, begins to collapse, and can ultimately fail to adequately support adjacent vertebrae. As a result, the neural element can become compressed within the neural foramen as well as the central canal of the spine leading to painful back conditions. Additionally, discogenic back pain, perhaps a less well understood condition, can also lead to painful back conditions as a result of disc degeneration.

The process of intervertebral disc degeneration occurs in all of us as we age and its treatment in symptomatic patients has significant socioeconomic impact. Many studies have shown that notochordal cells, the precursors of the disc, are no longer present after age 10. During embryogenesis, notochordal cells are believed to be responsible for the formation of spine and intervertebral disc, as well as for maintenance and metabolic control of the nucleus pulposus (NP) further in life. The relationship between loss of notochordal cells with age and the onset of disc degeneration can perhaps best be understood by the changes in biomechanics of the discs as a consequence of proteoglycan loss. The proteoglycans are the hydrophilic moiety of the intervertebral disc. These molecules are uniquely structured to hold water and therefore provide the cushioning quality of the intervertebral disc. It has been shown in recent studies that notochordal cells produce 1.5 fold more proteoglycans and extracellular matrix than terminally differentiated chondrocytes. As the notochordal cells differentiate to chondrocytes in the NP, less water holding proteoglycan matrix is available. A cascade of events ensues resulting in disc degeneration, desiccation and collapse. Consequently, the annulus fibrosis (AF) begins to fissure and cracks, contributing to a vicious cycle of disc degeneration potentially resulting in chronic lower back pain.

The intervertebral disc is comprised of an external AF made up primarily of lamellar bands of type I collagen that surrounds a soft gelatinous central NP made up primarily of type II collagen and a proteoglycan matrix. The proteoglycan moiety is a highly hydrophilic molecule capable of holding significant amounts of water. The water holding capacity of the NP, held within the confines of the intact AF, gives the intervertebral disc its unique function, particularly that of providing a mobile compressible distraction between adjacent vertebral bodies, and thus providing the unique flexibility of the spinal column.

Current treatments of degenerated disc are limited to surgery and disc ablation, both of which carry a significant degree of invasiveness and need for re-intervention. Although cellular therapies have shown promising results, these technologies are still not cleared by regulators for wide spread implementation. Additionally, these therapies, although addressing the need for regeneration do not alleviate the reduced blood flow to the discal area, nor do they address the underlying inflammatory processes that initiated the pathological process of disc degeneration. The current patent application addresses the hypoperfusion aspect by stimulating angiogenesis and the inflammatory aspect by potent immune modulation.

### SUMMARY OF THE INVENTION

A summary of the invention is provided below with respect to the following numbered aspects.

1. A method of preventing, reducing, or reversing disc degeneration comprising the steps of: a) obtaining a population of B regulatory cells; b) identifying a patient with disc degeneration; and c) administering said regulatory B cells into said patient with said disc degeneration.

2. The method of aspect 1, wherein said disc degeneration is characterized by loss of mechanical support afforded by intravertebral collagen.

3. The method of aspect 1, wherein said disc degeneration is characterized by loss of mechanical support afforded by intravertebral aggrecan.

4. The method of aspect 1, wherein said disc degeneration is characterized by dehydration of the nucleus pulposus.

5. The method of aspect 1, wherein said disc degeneration is associated with reduced concentrations of tissue inhibitor of metalloprotease (TIMP) activity.

6. The method of aspect 5, wherein said reduced TIMP activity is associated with lower expression of TIMP-1 protein as compared to a healthy age-matched control.

7. The method of aspect 5, wherein said reduced TIMP activity is associated with lower expression of TIMP-2 protein as compared to a healthy age-matched control.

8. The method of aspect 5, wherein said reduced TIMP activity is associated with lower expression of TIMP-3 protein as compared to a healthy age-matched control.

9. The method of aspect 5, wherein said reduced TIMP activity is associated with lower expression of TIMP-4 protein as compared to a healthy age-matched control.

10. The method of aspect 1, wherein said disc degeneration is characterized by upregulated expression of tissue degrading enzymes.

11. The method of aspect 10, wherein said tissue degrading enzymes are proteases.

12. The method of aspect 11, wherein said protease is urokinase type plasminogen

activator.

13. The method of aspect 11, wherein said protease is thrombin.

14. The method of aspect 11, wherein said protease is a matrix metalloprotease.

15. The method of aspect 14, wherein said matrix metalloprotease is MMP1.

16. The method of aspect 14, wherein said matrix metalloprotease is gelatinase A.

17. The method of aspect 14, wherein said matrix metalloprotease is stromelysin-1.

18. The method of aspect 14, wherein said matrix metalloprotease is matrilysin.

19. The method of aspect 14, wherein said matrix metalloprotease is neutrophil collagenase.

20. The method of aspect 14, wherein said matrix metalloprotease is gelatinase B.

21. The method of aspect 14, wherein said matrix metalloprotease is stromelysin-2.

22. The method of aspect 14, wherein said matrix metalloprotease is stromelysin-3.

23. The method of aspect 14, wherein said matrix metalloprotease is macrophage metalloelastase.

24. The method of aspect 14, wherein said matrix metalloprotease is collagenase 3.

25. The method of aspect 14, wherein said matrix metalloprotease is MT1-MMP.

26. The method of aspect 14, wherein said matrix metalloprotease is MT2-MMP.

27. The method of aspect 14, wherein said matrix metalloprotease is MT3-MMP.

28. The method of aspect 14, wherein said matrix metalloprotease is MT4-MMP.

29. The method of aspect 14, wherein said matrix metalloprotease is collagenase 4.

30. The method of aspect 14, wherein said matrix metalloprotease is stromelysin 4.

31. The method of aspect 14, wherein said matrix metalloprotease is enamelysin.

32. The method of aspect 14, wherein said matrix metalloprotease is X-MMP.

33. The method of aspect 14, wherein said matrix metalloprotease is CA-MMP.

34. The method of aspect 14, wherein said matrix metalloprotease is MMP22.

35. The method of aspect 14, wherein said matrix metalloprotease is MT5-MMP.

36. The method of aspect 14, wherein said matrix metalloprotease is MT6-MMP.

37. The method of aspect 14, wherein said matrix metalloprotease is Matrilysin-2.

38. The method of aspect 14, wherein said matrix metalloprotease is C-MMP.

39. The method of aspect 14, wherein said matrix metalloprotease is epilysin.

40. The method of aspect 1, wherein said disc degeneration is associated with increased inflammatory cell number in the intradiscal area.

41. The method of aspect 1, wherein said disc degeneration is associated with increased inflammatory cell number in the peri-discal area.

42. The method of aspects 40 and 41, wherein said inflammatory cells are neutrophils.

43. The method of aspect 42, wherein said neutrophils have an increased resistance to apoptosis as compared to neutrophils obtained from a healthy age-matched control.

44. The method of aspect 42, wherein said neutrophils have an increased propensity to produce TNF-alpha as compared to neutrophils obtained from a healthy age-matched control.

45. The method of aspect 42, wherein said neutrophils have an increased propensity to produce MMP1 as compared to neutrophils obtained from a healthy age-matched control.

46. The method of aspect 42, wherein said neutrophils have an increased propensity to produce MMP3 as compared to neutrophils obtained from a healthy age-matched control.

47. The method of aspect 42, wherein said neutrophils have an increased propensity to produce MMP7 as compared to neutrophils obtained from a healthy age-matched control.

48. The method of aspect 42, wherein said neutrophils have an increased propensity to produce MMP9 as compared to neutrophils obtained from a healthy age-matched control.

49. The method of aspect 42, wherein said neutrophils have an increased propensity to produce MMP11 as compared to neutrophils obtained from a healthy age-matched control.

50. The method of aspect 42, wherein said neutrophils have an increased propensity to produce MMP15 as compared to neutrophils obtained from a healthy age-matched control.

51. The method of aspect 42, wherein said neutrophils have an increased propensity to produce lymphotoxin as compared to neutrophils obtained from a healthy age-matched control.

52. The method of aspect 42, wherein said neutrophils have an increased propensity to produce alpha 1 antitrypsin as compared to neutrophils obtained from a healthy age-matched control.

53. The method of aspect 42, wherein said neutrophils have an increased propensity to produce interferon alpha as compared to neutrophils obtained from a healthy age-matched control.

54. The method of aspect 42, wherein said neutrophils have an increased propensity to produce interleukin-1 beta as compared to neutrophils obtained from a healthy age-matched control.

55. The method of aspect 42, wherein said neutrophils have an increased propensity to produce interleukin-2 as compared to neutrophils obtained from a healthy age-matched control.

56. The method of aspect 42, wherein said neutrophils have an increased propensity to produce interleukin-5 as compared to neutrophils obtained from a healthy age-matched control.

57. The method of aspect 42, wherein said neutrophils have an increased propensity to produce interleukin-6 as compared to neutrophils obtained from a healthy age-matched control.

58. The method of aspect 42, wherein said neutrophils have an increased propensity to produce interleukin-8 as compared to neutrophils obtained from a healthy age-matched control.

59. The method of aspect 42, wherein said neutrophils have an increased propensity to produce interleukin-9 as compared to neutrophils obtained from a healthy age-matched control.

60. The method of aspect 42, wherein said neutrophils have an increased propensity to produce interleukin-11 as compared to neutrophils obtained from a healthy age-matched control.

61. The method of aspect 42, wherein said neutrophils have an increased propensity to produce interleukin-12 as compared to neutrophils obtained from a healthy age-matched control.

62. The method of aspect 42, wherein said neutrophils have an increased propensity to produce interleukin-14 as compared to neutrophils obtained from a healthy age-matched control.

63. The method of aspect 42, wherein said neutrophils have an increased propensity to produce interleukin-15 as compared to neutrophils obtained from a healthy age-matched control.

64. The method of aspect 42, wherein said neutrophils have an increased propensity to produce interleukin-17 as compared to neutrophils obtained from a healthy age-matched control.

65. The method of aspect 42, wherein said neutrophils have an increased propensity to produce interleukin-17A as compared to neutrophils obtained from a healthy age-matched control.

66. The method of aspect 42, wherein said neutrophils have an increased propensity to produce interleukin-17C as compared to neutrophils obtained from a healthy age-matched control.

67. The method of aspect 42, wherein said neutrophils have an increased propensity to produce interleukin-17F as compared to neutrophils obtained from a healthy age-matched control.

68. The method of aspect 42, wherein said neutrophils have an increased propensity to produce interleukin-18 as compared to neutrophils obtained from a healthy age-matched control.

70. The method of aspect 42, wherein said neutrophils have an increased propensity to produce interleukin-21 as compared to neutrophils obtained from a healthy age-matched control.

71. The method of aspect 42, wherein said neutrophils have an increased propensity to produce interleukin-22 as compared to neutrophils obtained from a healthy age-matched control.

72. The method of aspect 1, wherein said B regulatory cells are generated by culture of B cell progenitors with tolerogenic dendritic cells.

73. The method of aspect 72, wherein said tolerogenic dendritic cells possess a reduced amount of CD86 as compared to a dendritic cell in basal conditions.

74. The method of aspect 72, wherein said tolerogenic dendritic cells possess a reduced amount of CD5 as compared to a dendritic cell in basal conditions.

75. The method of aspect 1, wherein said B regulatory cells are generated by culture of B cell progenitors with mesenchymal stem cells.

76. The method of aspect 75, wherein said mesenchymal stem cells are cultured in the presence of a histone deacetylase inhibitor.

77. The method of aspect 76, wherein said 76, wherein said histone deacetylase inhibitor is valproic acid.

78. The method of aspect 76, wherein said 76, wherein said histone deacetylase inhibitor is butyrate.

79. The method of aspect 76, wherein said 76, wherein said histone deacetylase inhibitor is pterostilbene.

80. The method of aspect 76, wherein said 76, wherein said histone deacetylase inhibitor is resveratrol.

81. The method of aspect 75, wherein said mesenchymal stem cells are cultured in the presence of G-CSF.

82. The method of aspect 75, wherein said mesenchymal stem cells are cultured in the presence of GM-CSF.

83. The method of aspect 75, wherein said mesenchymal stem cells are cultured in the presence of EGF-1.

84. The method of aspect 75, wherein said mesenchymal stem cells are cultured in the presence of HGF-1.

85. The method of aspect 75, wherein said mesenchymal stem cells are cultured in the presence of IGF-1.

86. The method of aspect 75, wherein said mesenchymal stem cells are cultured in the presence of amphiregulin.

87. The method of aspect 75, wherein said mesenchymal stem cells are cultured in the presence of interleukin-10.

88. The method of aspect 75, wherein said mesenchymal stem cells are cultured in the presence of HGF-1 and G-CSF.

89. The method of aspect 75, wherein said mesenchymal stem cells are cultured in the presence of HGF-1 and GM-CSF.

90. The method of aspect 75, wherein said mesenchymal stem cells are cultured in the presence of a toll like receptor agonist.

91. The method of aspect 90, wherein said toll like receptor agonist is an activator of toll like receptor 2.

92. The method of aspect 91, wherein said agonist of said toll like receptor 2 is Pam3CSK4.

93. The method of aspect 91, wherein said agonist of said toll like receptor 2 is P3C4.

94. The method of aspect 90, wherein said toll like receptor agonist is an activator of toll like receptor 3.

95. The method of aspect 94, wherein said activator of said toll like receptor 3 is double stranded RNA.

96. The method of aspect 94, wherein said activator of said toll like receptor 3 is poly IC.

97. The method of aspect 94, wherein said activator of said toll like receptor 3 is poly LC.

98. The method of aspect 94, wherein said activator of said toll like receptor 3 is autologous cellular lysate.

99. The method of aspect 90, wherein said toll like receptor agonist activates toll like receptor 9.

100. The method of aspect 99, wherein said toll like receptor 9 is activated by administration of CpG motif containing DNA

101. The method of aspect 1, wherein an immune modulator capable of increasing B regulatory cell numbers and/or B regulatory cell activity is administered together with said perinatal tissue derived exosomes.

102. The method of aspect 102, wherein said B regulatory cells express AIRE.

103. The method of aspect 102, wherein said B regulatory cells express GITR ligand.

104. The method of aspect 102, wherein said B regulatory cells express membrane bound TGF-beta.

105. The method of aspect 102, wherein said B regulatory cells express membrane Fas ligand.

106. The method of aspect 102, wherein said B regulatory cells express IL-10 receptor.

107. The method of aspect 102, wherein said B regulatory cells express IL-35 receptor.

108. The method of aspect 102, wherein said B regulatory cells express membrane bound CTLA4.

109. The method of aspect 102, wherein said B regulatory cells express membrane bound CD25.

110. The method of aspect 102, wherein said B regulatory cells express TIM3.

111. The method of aspect 102, wherein said B regulatory cells possess ability to block B cell proliferation.

112. The method of aspect 111, wherein said T cell proliferation is stimulated by activation of T cell receptor zeta chain.

113. The method of aspect 111, wherein said T cell proliferation is stimulated by activation of T cell receptor.

114. The method of aspect 111, wherein said T cell proliferation is stimulated by activation of CD3.

115. The method of aspect 111, wherein said T cell proliferation is stimulated by activation of CD3 and a costimulatory molecule.

116. The method of aspect 115, wherein said costimulatory molecule is CD28.

117. The method of aspect 111, wherein said T cell proliferation is stimulated by interleukin-7.

118. The method of aspect 111, wherein said T cell proliferation is stimulated by interleukin-15.

119. The method of aspect 111, wherein said T cell proliferation is stimulated by exposure to a mitogen.

120. The method of aspect 119, wherein said mitogen is a lectin.

121. The method of aspect 120, wherein said lectin is phytohemagglutinin.

122. The method of aspect 120, wherein said lectin is pokeweek mitogen.

123. The method of aspect 102, wherein said B regulatory cells inhibit production of Th1 cytokines from an activated T cell population.

124. The method of aspect 123, wherein said Th1 cytokine is interferon gamma.

125. The method of aspect 123, wherein said Th1 cytokine is interferon alpha.

126. The method of aspect 123, wherein said Th1 cytokine is interferon beta.

127. The method of aspect 123, wherein said Th1 cytokine is interleukin-7.

128. The method of aspect 123, wherein said Th1 cytokine is interleukin-12.

129. The method of aspect 123, wherein said Th1 cytokine is interleukin-15.

130. The method of aspect 123, wherein said Th1 cytokine is interleukin-18.

131. The method of aspect 102, wherein said B regulatory cells suppress generation of Th17 cells.

132. The method of aspect 102, wherein said B regulatory cells suppress generation of Th9 cells.

133. The method of aspect 102, wherein said B regulatory cells suppress NK cell activity.

134. The method of aspect 102, wherein said B regulatory cells enhance generation of myeloid suppressor cells.

135. The method of aspect 134, wherein said myeloid suppressor cells are capable of differentiating into neutrophils.

136. The method of aspect 134, wherein said myeloid suppressor cells are capable of differentiating into monocytes.

137. The method of aspect 134, wherein said myeloid suppressor cells express STAT3.

138. The method of aspect 134, wherein said myeloid suppressor cells are capable of producing reptimed.

139. The method of aspect 134, wherein said myeloid suppressor cells are capable of producing PGE2.

140. The method of aspect 134, wherein said myeloid suppressor cells are capable of producing nitric oxide.

141. The method of aspect 134, wherein said myeloid suppressor cells are capable of producing TGF-beta.

142. The method of aspect 134, wherein said myeloid suppressor cells are capable of producing leukemia inhibitory factor.

143. The method of aspect 134, wherein said myeloid suppressor cells are capable of producing interleukin-10.

144. The method of aspect 134, wherein said myeloid suppressor cells are capable of producing interleukin-35.

145. The method of aspect 101, wherein said B regulatory cells are stimulated by administration of anti-CD3 antibody.

146. The method of aspect 145, wherein said anti-CD3 antibody is administered together with low dose IL-2.

147. The method of aspect 145, wherein said anti-CD3 antibody levels of administration are determined by quantification of T regulatory cells in circulation after administration.

148. The method of aspect 147, wherein said quantification of said B regulatory cells is performed by assessment of FoxP3.

149. The method of aspect 148, wherein assessment of FoxP3 is performed by flow cytometry.

150. The method of aspect 147, wherein said assessment of B regulatory cells is performed by quantification of GITR ligand in the blood.

151. The method of aspect 150, wherein said assessment of GITR ligand is performed by flow cytometry and/or mass cytometry.

152. The method of aspect 101, wherein said B regulatory cells are stimulated by administration of resveratrol.

153. The method of aspect 101, wherein said B regulatory cells are stimulated by administration of pterostilbene.

154. The method of aspect 101, wherein said B regulatory cells are stimulated by administration of quercetin.

155. The method of aspect 101, wherein said B regulatory cells are stimulated by administration of vitamin D3.

156. The method of aspect 101, wherein said B regulatory cells are stimulated by administration of ivermectin.

157. The method of aspect 101, wherein said B regulatory cells are stimulated by administration of n-acetylcysteine.

158. The method of aspect 101, wherein said B regulatory cells are stimulated by administration of IVIG.

159. The method of aspect 158, wherein said IVIG is administered together with low dose interleukin-2.

160. The method of aspect 159, wherein said IVIG and low dose interleukin-2 is administered together with mesenchymal stem cells.

161. The method of aspect 160, wherein said mesenchymal stem cells are derived from perivascular tissue.

162. The method of aspect 160, wherein said perivascular tissue is associated with enhanced concentrations of high molecular weight hyaluronic acid as compared to other tissues.

163. The method of aspect 160, wherein said mesenchymal stem cells are derived from omental tissue.

164. The method of aspect 160, wherein said mesenchymal stem cells are derived from placental tissue.

165. The method of aspect 160, wherein said mesenchymal stem cells are derived from testicular tissue.

166. The method of aspect 160, wherein said mesenchymal stem cells are derived from endometrial tissue.

167. The method of aspect 160, wherein said mesenchymal stem cells are derived from fallopian tubes.

168. The method of aspect 160, wherein said mesenchymal stem cells are derived from bone marrow.

169. The method of aspect 160, wherein said mesenchymal stem cells are derived from peripheral blood.

170. The method of aspect 160, wherein said mesenchymal stem cells are derived from mobilized peripheral blood.

171. The method of aspect 170, wherein said mobilization of peripheral blood is accomplished by administration of a sympathetic nervous system stimulant.

172. The method of aspect 170, wherein said mobilization of peripheral blood is accomplished by administration of G-CSF.

173. The method of aspect 170, wherein said mobilization of peripheral blood is accomplished by administration of GM-CSF.

174. The method of aspect 170, wherein said mobilization of peripheral blood is accomplished by administration of M-CSF.

175. The method of aspect 170, wherein said mobilization of peripheral blood is accomplished by administration of flt-3 ligand.

176. The method of aspect 170, wherein said mobilization of peripheral blood is accomplished by administration of HGF.

177. The method of aspect 170, wherein said mobilization of peripheral blood is accomplished by administration of IGF.

178. The method of aspect 170, wherein said mobilization of peripheral blood is accomplished by administration of FGF-1.

179. The method of aspect 170, wherein said mobilization of peripheral blood is accomplished by administration of FGF-2.

180. The method of aspect 170, wherein said mobilization of peripheral blood is accomplished by administration of GDF-11.

181. The method of aspect 170, wherein said mobilization of peripheral blood is accomplished by administration of leukemia inhibitor factor.

182. The method of aspect 170, wherein said mobilization of peripheral blood is accomplished by administration of BDNF.

183. The method of aspect 170, wherein said mobilization of peripheral blood is accomplished by administration of NGF.

184. The method of aspect 160, wherein said mesenchymal stem cells are derived from deciduous teeth.

185. The method of aspect 160, wherein said mesenchymal stem cells are derived from menstrual blood.

186. The method of aspect 160, wherein said mesenchymal stem cells are derived from amniotic membrane.

187. The method of aspect 160, wherein said mesenchymal stem cells are derived from amniotic fluid.

188. The method of aspect 160, wherein said mesenchymal stem cells are derived from cerebral spinal fluid.

189. The method of aspect 160, wherein said mesenchymal stem cells are derived from trophoblastic tissue.

190. The method of aspect 160, wherein said mesenchymal stem cells are derived from dermal tissue.

191. The method of aspect 160, wherein said mesenchymal stem cells are derived from hair follicle.

192. The method of aspect 160, wherein said mesenchymal stem cells are derived from lymphatic tissue.

193. The method of aspect 160, wherein said mesenchymal stem cells are pre-treated with one or more agents or conditions to increase ability to home to an area of inflammation.

194. The method of aspect 193, wherein said condition to increase homing of said cell to area of inflammation increases expression of CXCR4.

195. The method of aspect 193, wherein said mesenchymal stem cells are exposed to hypoxia.

196. The method of aspect 193, wherein said mesenchymal stem cells are exposed to acidosis.

197. The method of aspect 193, wherein said mesenchymal stem cells are exposed to hyperthermia.

198. The method of aspect 193, wherein said mesenchymal stem cells are treated with TNF-alpha.

199. The method of aspect 193, wherein said mesenchymal stem cells are treated with interferon gamma.

200. The method of aspect 193, wherein said mesenchymal stem cells are treated with one or more stimulators of the enzyme indolamine 2,3 dioxygenase.

201. The method of aspect 1, wherein said B regulatory cells are generated from a pluripotent stem cell source.

202. The method of aspect 201, wherein said pluripotent stem cell source useful for generating B regulatory cells is a somatic cell nuclear transfer derived cell.

203. The method of aspect 201, wherein said pluripotent stem cell source useful for generating B regulatory cells is a parthenogenesis derived stem cell.

204. The method of aspect 201, wherein said pluripotent stem cell source useful for generating B regulatory cells is an induced pluripotent stem cell.

205. The method of aspect 204, wherein said induced pluripotent derived stem cell is generated from an immature cellular source.

206. The method of aspect 205, wherein said immature stem cell source is a hematopoietic stem cell.

207. The method of aspect 206, wherein said hematopoietic stem cell is capable of recapitulating multilineage hematopoietic reconstitution in an immune deficient host.

208. The method of aspect 207, wherein said hematopoietic stem cell expresses CD34.

209. The method of aspect 207, wherein said hematopoietic stem cell expresses CD133.

210. The method of aspect 207, wherein said hematopoietic stem cell expresses aldehyde dehydrogenase.

211. The method of aspect 207, wherein said hematopoietic stem cell expresses c-kit.

212. The method of aspect 207, wherein said hematopoietic stem cell expresses interleukin-7 receptor.

213. The method of aspect 207, wherein said hematopoietic stem cell expresses c-met.

214. The method of aspect 207, wherein said hematopoietic stem cell expresses rhodamine 231 efflux transporter.

215. The method of aspect 207, wherein said hematopoietic stem cell expresses interleukin-3 receptor.

216. The method of aspect 207, wherein said hematopoietic stem cell expresses interleukin-6 receptor.

217. The method of aspect 207, wherein said hematopoietic stem cell expresses interleukin-11 receptor.

218. The method of aspect 207, wherein said hematopoietic stem cell expresses leukemia inhibitor factor receptor.

219. The method of aspect 207, wherein said hematopoietic stem cell expresses Fas ligand

220. The method of aspect 207, wherein said hematopoietic stem cell expresses CD93.

221. The method of aspect 205, wherein said immature stem cell source is an amniotic fluid mesenchymal stem cell.

222. The method of aspect 205, wherein said immature stem cell source is an amniotic membrane mesenchymal stem cell.

223. The method of aspect 205, wherein said immature stem cell source is an umbilical cord blood mesenchymal stem cell.

224. The method of aspect 205, wherein said immature stem cell source is an umbilical cord tissue mesenchymal stem cell.

225. The method of aspect 205, wherein said immature stem cell source is an adipose mesenchymal stem cell.

226. The method of aspect 205, wherein said immature stem cell source is an endometrial mesenchymal stem cell.

227. The method of aspect 205, wherein said immature stem cell source is a hepatic mesenchymal stem cell.

228. The method of aspect 205, wherein said immature stem cell source is a peripheral blood mesenchymal stem cell.

229. The method of aspect 205, wherein said immature stem cell source is a bone marrow stem cell.

230. The method of aspect 204, wherein said induced pluripotent stem cell is generated by transfection with one or more of the following: a) OCT4; b) NANOG; c) Lin28; d) Sox-2; e) c-myc; f) KLF4; g) Pim-1 and h) RAS.

231. The method of aspect 230, wherein said transfection is performed in the presence of valproic acid.

232. The method of aspect 231, wherein said valproic acid is added at a concentration and time period sufficient to increase expression of c-met in a CD133 hematopoietic stem cell by more than 10% as compared to baseline.

233. The method of aspect 231, wherein said valproic acid is added at a concentration and time period sufficient to increase expression of c-met in a CD133 hematopoietic stem cell by more than 25% as compared to baseline.

234. The method of aspect 231, wherein said valproic acid is added at a concentration and time period sufficient to increase expression of c-met in a CD133 hematopoietic stem cell by more than 100% as compared to baseline.

235. The method of aspect 231, wherein said valproic acid is added at a concentration and time period sufficient to increase expression of c-kit in a CD133 hematopoietic stem cell by more than 10% as compared to baseline.

236. The method of aspect 231, wherein said valproic acid is added at a concentration and time period sufficient to increase expression of c-kit in a CD133 hematopoietic stem cell by more than 25% as compared to baseline.

237. The method of aspect 231, wherein said valproic acid is added at a concentration and time period sufficient to increase expression of c-kit in a CD133 hematopoietic stem cell by more than 100% as compared to baseline.

238. The method of aspect 231, wherein said valproic acid is added at a concentration and time period sufficient to increase expression of interleukin-3 receptor in a CD133 hematopoietic stem cell by more than 10% as compared to baseline.

239. The method of aspect 231, wherein said valproic acid is added at a concentration and time period sufficient to increase expression of interleukin-3 receptor in a CD133 hematopoietic stem cell by more than 25% as compared to baseline.

240. The method of aspect 231, wherein said valproic acid is added at a concentration and time period sufficient to increase expression of interleukin-7 receptor in a CD133 hematopoietic stem cell by more than 10% as compared to baseline.

241. The method of aspect 231, wherein said valproic acid is added at a concentration and time period sufficient to increase expression of interleukin-7 receptor in a CD133 hematopoietic stem cell by more than 25% as compared to baseline.

242. The method of aspect 231, wherein said valproic acid is added at a concentration and time period sufficient to increase expression of interleukin-7 receptor in a CD133 hematopoietic stem cell by more than 100% as compared to baseline.

243. The method of aspect 231, wherein said valproic acid is added at a concentration and time period sufficient to increase expression of interleukin-11 receptor in a CD133 hematopoietic stem cell by more than 10% as compared to baseline.

244. The method of aspect 231, wherein said valproic acid is added at a concentration and time period sufficient to increase expression of interleukin-11 receptor in a CD133 hematopoietic stem cell by more than 25% as compared to baseline.

245. The method of aspect 231, wherein said valproic acid is added at a concentration and time period sufficient to increase expression of interleukin-11 receptor in a CD133 hematopoietic stem cell by more than 100% as compared to baseline.

246. The method of aspect 231, wherein said valproic acid is added at a concentration and time period sufficient to increase expression of thrombopoietin receptor in a CD133 hematopoietic stem cell by more than 10% as compared to baseline.

247. The method of aspect 231, wherein said valproic acid is added at a concentration and time period sufficient to increase expression of thrombopoietin receptor in a CD133 hematopoietic stem cell by more than 25% as compared to baseline.

248. The method of aspect 231, wherein said valproic acid is added at a concentration and time period sufficient to increase expression of thrombopoietin receptor in a CD133 hematopoietic stem cell by more than 100% as compared to baseline.

249. The method of aspect 231, wherein said valproic acid is added at a concentration and time period sufficient to increase expression of hTERT in a CD133 hematopoietic stem cell by more than 10% as compared to baseline.

250. The method of aspect 231, wherein said valproic acid is added at a concentration and time period sufficient to increase expression of hTERT in a CD133 hematopoietic stem cell by more than 25% as compared to baseline.

251. The method of aspect 231, wherein said valproic acid is added at a concentration and time period sufficient to increase expression of hTERT in a CD133 hematopoietic stem cell by more than 100% as compared to baseline.

### DETAILED DESCRIPTION OF THE INVENTION

The invention provides the use of B regulatory cells for treatment of disc degenerative disease. Broadly speaking, the invention describes the administration of B regulatory cells as a means of suppressing localized inflammation in the intervertebral disc, as well as in the perispinal space. By reducing inflammation, the B regulatory cells provide an environment conducive to regeneration. For example, the administration of B regulatory cells allows for activation and function of endogenous stem cells, as well as exogenously administered stem cells. The invention further provides for the administration of B regulatory cells as a stimulatory of angiogenesis. In one embodiment the invention provides the previously unknown ability of B regulatory cells to stimulate angiogenesis. Stimulation of angiogenesis in the perispinal area is important for the treatment of conditions such as lumbar ischemia, which is believed to be one of the causes of disc degeneration and/or disc pain.

As used herein, the term “mammalian cells” in reference to transfected or transduced cells includes all types of mammalian cells, in particular human cells, including but not limited to connective tissue cells such as fibroblasts or chondrocytes, or stem cells, and in particular human embryonic kidney cells, and further in particular, human embryonic kidney 293 cells, or epithelial cells.

As used herein, the term “connective tissue” is any tissue that connects and supports other tissues or organs, and includes but is not limited to a ligament, a cartilage, a tendon, a bone, and a synovium of a mammalian host.

As used herein, the term “connective tissue cell” or “cell of a connective tissue” include cells that are found in the connective tissue, such as fibroblasts, cartilage cells (chondrocytes), and bone cells (osteoblasts/osteocytes), which secrete collagenous extracellular matrix, as well as fat cells (adipocytes) and smooth muscle cells. Preferably, the connective tissue cells are fibroblasts, chondrocytes, or bone cells. More preferably, the connective tissue cells are chondrocytes cells. It will be recognized that the invention can be practiced with a mixed culture of connective tissue cells, as well as cells of a single type. Preferably, the connective tissue cell does not cause a negative immune response when injected into the host organism. It is understood that allogeneic cells may be used in this regard, as well as autologous cells for cell-mediated gene therapy or somatic cell therapy.

As used herein, “connective tissue cell line” includes a plurality of connective tissue cells originating from a common parent cell.

As used herein, “hyaline cartilage” refers to the connective tissue covering the joint surface. By way of example only, hyaline cartilage includes, but is not limited to, articular cartilage, costal cartilage, and nose cartilage.

In particular, hyaline cartilage is known to be self-renewing, responds to alterations, and provides stable movement with less friction. Hyaline cartilage found even within the same joint or among joints varies in thickness, cell density, matrix composition and mechanical properties, yet retains the same general structure and function. Some of the functions of hyaline cartilage include surprising stiffness to compression, resilience, and exceptional ability to distribute weight loads, ability to minimize peak stress on subchondral bone, and great durability.

Grossly and histologically, hyaline cartilage appears as a slick, firm surface that resists deformation. The extracellular matrix of the cartilage comprises chondrocytes, but lacks blood vessels, lymphatic vessels or nerves. An elaborate, highly ordered structure that maintains interaction between chondrocytes and the matrix serves to maintain the structure and function of the hyaline cartilage, while maintaining a low level of metabolic activity. The reference O'Driscoll, J. Bone Joint Surg., 80A: 1795-1812, 1998 describes the structure and function of hyaline cartilage in detail, which is incorporated herein by reference in its entirety.

As used herein, “injectable” composition refers to a composition that excludes various three-dimensional scaffold, framework, mesh or felt structure, which may be made of any material or shape that allows cells to attach to it and allows cells to grow in more than one layer, and which structure is generally implanted, and not injected. In one embodiment, the injection method of the invention is typically carried out by a syringe. However, any mode of injecting the composition of interest may be used. For instance, catheters, sprayers, or temperature dependent polymer gels also may be used.

As used herein, “juvenile chondrocyte” refers to chondrocyte obtained from a human being who is less than two years old. Typically, the chondrocyte is obtained from preferably the hyaline cartilage region of an extremity of the body, such as a finger, nose, ear lobe and so forth. Juvenile chondrocytes may be used as donor chondrocytes for allogeneic treatment of defected or injured intervertebral disc.

As used herein, the term “mammalian host” includes members of the animal kingdom including but not limited to human beings.

As used herein, “mixed cell” or a “mixture of cells” or “cell mixture” refers to the combination of a plurality of cells that include a first population of cells that are transfected or transduced with a gene of interest and a second population of cells that are untransduced.

In one embodiment of the invention, mixed cells may refer to the combination of a plurality of cells that include cells that have been transfected or transduced with a gene or DNA encoding a member of the transforming growth factor β superfamily and cells that have not been transfected or transduced with a gene encoding a member of the transforming growth factor β superfamily. Typically, the ratio of cells that have not been transfected or transduced with a gene encoding a member of the transforming growth factor β superfamily to cells that have been transfected or transduced with a TGF superfamily gene may be in the range of about 3-20 to 1. The range may include about 3-10 to 1. In particular, the range may be about 10 to 1 in terms of the number of cells. However, it is understood that the ratio of these cells should not be necessarily fixed to any particular range so long as the combination of these cells is effective to treat injured intervertebral disc by slowing or retarding degeneration of defected intervertebral disc.

As used herein, “non-disc chondrocyte” refers to chondrocytes isolated from any part of the body except for intervertebral disc cartilage tissue. Non-disc chondrocytes of the present invention may be used for allogeneic transplantation or injection into a patient to treat defected or injured intervertebral disc.

As used herein, the term “patient” includes members of the animal kingdom including but not limited to human beings.

As used herein, the term “primed” cell refers to cells that have been activated or changed to express certain genes.

As used herein, “slowing” or “prevention” of intervertebral disc degeneration refers to the retention of volume of intervertebral disc or height of the disc over time compared with the volume or height level that would normally be found at the site of injury leading to normal degeneration over a given time. This may mean a percentage increase of volume or height, such as about 10%, 20%, 30%, 40%, 50%, 60%, 70%, 80%, or 90% compared with the normal expected degeneration levels at a given time, or may mean lessening of damage or depletion of volume or height of the intervertebral disc at the locus.

As used herein, the “transforming growth factor-β (TGF-β) superfamily” encompasses a group of structurally related proteins, which affect a wide range of differentiation processes during embryonic development. The family includes, Müllerian inhibiting substance (MIS), which is required for normal male sex development (Behringer, et al., Nature, 345:167, 1990), Drosophila decapentaplegic (DPP) gene product, which is required for dorsal-ventral axis formation and morphogenesis of the imaginal discs (Padgett, et al., Nature, 325:81-84, 1987), the Xenopus Vg-1 gene product, which localizes to the vegetal pole of eggs (Weeks, et al., Cell, 51:861-867, 1987), the activins (Mason, et al., Biochem, Biophys. Res. Commun., 135:957-964, 1986), which can induce the formation of mesoderm and anterior structures in Xenopus embryos (Thomsen, et al., Cell, 63:485, 1990), and the bone morphogenetic proteins (BMP's, such as BMP-2, 3, 4, 5, 6 and 7, osteogenin, OP-1) which can induce de novo cartilage and bone formation (Sampath, et al., J. Biol. Chem., 265:13198, 1990). The TGF-β gene products can influence a variety of differentiation processes, including adipogenesis, myogenesis, chondrogenesis, hematopoiesis, and epithelial cell differentiation (for a review, see Massague, Cell 49:437, 1987), which is incorporated herein by reference in its entirety.

The proteins of the TGF-β family are initially synthesized as a large precursor protein, which subsequently undergoes proteolytic cleavage at a cluster of basic residues approximately 110-140 amino acids from the C-terminus. The C-terminal regions of the proteins are all structurally related and the different family members can be classified into distinct subgroups based on the extent of their homology. Although the homologies within particular subgroups range from 70% to 90% amino acid sequence identity, the homologies between subgroups are significantly lower, generally ranging from only 20% to 50%. In each case, the active species appears to be a disulfide-linked dimer of C-terminal fragments. For most of the family members that have been studied, the homodimeric species has been found to be biologically active, but for other family members, like the inhibins (Ung, et al., Nature, 321:779, 1986) and the TGF-β's (Cheifetz, et al., Cell, 48:409, 1987), heterodimers have also been detected, and these appear to have different biological properties than the respective homodimers.

Members of the superfamily of TGF-β genes include TGF-β3, TGF-β2, TGF-β4 (chicken), TGF-β1, TGF-β5 (Xenopus), BMP-2, BMP-4, Drosophila DPP, BMP-5, BMP-6, Vgr1, OP-1/BMP-7, Drosophila 60A, GDF-1, Xenopus Vgf, BMP-3, Inhibin-βA, Inhibin-PB, Inhibin-α, and MIS. These genes are discussed in Massague, Ann. Rev. Biochem. 67:753-791, 1998, which is incorporated herein by reference in its entirety.

Preferably, the member of the superfamily of TGF-β genes is TGF-β1, TGF-β2, TGF-β3, BMP-2, BMP-3, BMP-4, BMP-5, BMP-6, or BMP-7.

In one embodiment the invention provides generation of B regulatory cells from B cells. Additionally, in other embodiments, the invention provides the utilization of induced pluripotent stem cells for stimulation of B regulatory cell production. In the present invention, “pluripotent stem cells” refer to stem cells produced from the inner cell mass of an embryo of an animal in the blastocyst stage or cells having phenotypes similar to those cells. Specifically, pluripotent stem cells induced in the present invention are cells that express alkaline phosphatase which is an indicator of ES-like cells. Here, “ES-like cells” indicate pluripotent stem cells having properties and/or morphologies that are similar to ES cells. Furthermore, preferably, when pluripotent stem cells are cultured, they form flat colonies containing cells with a higher proportion of nucleus volume than cytoplasm. Culturing may be carried out suitably with a feeder. Moreover, while cultured cells such as MEF stop proliferating in a few weeks, pluripotent stem cells can be passaged for a long period of time, and this can be confirmed based on their proliferative character that is not lost even when they are passaged, for example, 15 times or more, preferably 20 times or more, 25 times or more, 30 times or more, 35 times or more, or 40 times or more every three days. Furthermore, pluripotent stem cells preferably express endogenous OCT3/4 or Nanog, or more preferably, they express both of them. Furthermore, pluripotent stem cells preferably express TERT, and show telomerase activity (activity to synthesize telomeric repeat sequences). Moreover, pluripotent stem cells preferably have the ability to differentiate into three germ layers (the endoderm, mesoderm, and ectoderm) (this can be confirmed, for example, during teratoma formation and/or embryoid body formation). More preferably, pluripotent stem cells produce germline chimera when they are transplanted into blastocysts, Pluripotent stem cells capable of germline transmission are called germline-competent pluripotent stem cells. Confirmation of these phenotypes can be carried out by known methods (WO 2007/69666; Ichisaka T. et al., Nature 448 (7151):313-7, 2007).

Techniques for the generation of pluripotent stem cells such as iPSC are known in the art, in one embodiment said cells are created by transfection of genes such as OCT4 into the cells, alone, or along with other factors. This is typically performed using viral vectors, one idea viral vector is the Sendai virus. The genome of wild-type Sendai virus includes a short 3′ leader region followed by a nucleocapsid (NP) gene, a phospho (P) gene, a matrix (M) gene, a fusion (F) gene, a hemagglutinin-neuraminidase (HN) gene, and a large (L) gene, and then a short 5′ trailer region, in this order. Viral genes can be positioned in this order in Sendai virus vectors as well. Production of recombinant vectors comparable to wild-type viruses, and various mutant vectors are already known. Furthermore, it has been shown that gene delivery is possible using the RNP alone without its envelope (WO 00/70055). Therefore, reprogramming can be carried out using Sendai virus RNP as a virus vector.

Sendai viruses are sufficiently functional as vectors if they carry the NP gene, P gene and L gene; and they can replicate genome in cells and express the loaded foreign genes (KLF, OCT, SOX, and such). In a Sendai virus carrying the three genes, NP gene, P gene and L gene as viral genes, the set of KLF, OCT, and SOX genes is inserted, for example, between the P gene and the L gene. In a vector containing the M gene, the set of KLF, OCT, and SOX genes is inserted, for example, between the P gene and the M gene (WO 97/16539). When the F gene is included in an M gene-deleted Sendai virus vector, the set of KLF, OCT, and SOX genes is inserted between the P gene and the F gene (WO 00/70070). For the M and F gene-deleted Sendai virus vector, the position of insertion is between the P gene and the HN gene; and for the F, M, and HN gene-deleted Sendai virus vector, the position of insertion is between the P gene and the L gene (WO 2003/025570, WO 2006/137517), Viral gene-deleted vectors are preferable since they are very safe. In the present invention, a vector having at least the F gene deletion can be preferably used. Moreover, for a vector containing the KLF gene but not containing the OCT gene and the SOX gene, the KLF gene is inserted for example upstream of the NP gene (between the 3′ leader sequence and the NP gene). The above-mentioned Sendai virus vector containing the KLF, OCT, and SOX genes and the Sendai virus vector containing the KLF gene but not containing the OCT gene and the SOX gene can be appropriately used by the two of them for gene delivery in reprogramming, but they are more preferably used in reprogramming that further involves introduction of the MYC gene. A vector expressing the Glis1 gene (Maekawa et al., Nature, 474:225-229, 2011) instead of the MYC gene may also be combined. The MYC gene or Glis1 gene can be inserted into the above-mentioned Sendai virus vector containing the KLF, OCT and SOX genes or the Sendai virus vector containing the KLF gene but not the OCT gene and the SOX gene, but it may also be used after insertion into a different vector. When inserting the MYC gene or Glis1 gene into a different vector, desired vectors such as plasmids, virus vectors, and non-virus vectors (for example, liposomes) can be used. Examples of virus vectors include Adenovirus vectors and retrovirus vectors, but are not limited thereto. More preferably, the MYC gene or Glis1 gene is inserted into a Sendai virus vector. In the present invention, the above-mentioned Sendai virus vector containing the KLF, OCT, and SOX genes and the Sendai virus vector containing the KLF gene but not the OCT gene and the SOX gene are preferably used in combination with a different Sendai virus vector inserted with the MYC gene or (Hist. gene. The above-mentioned Sendai virus vector can be used as the starting Sendai virus vector for insertion of the MYC gene or Glis1 gene.

When inserting the MYC gene or Glis1 gene into a Sendai virus genome, a desirable site can be selected for the position of gene insertion in the vector. For example, the MYC gene or Glis1 gene is preferably positioned toward the rear of the minus-strand RNA genome (5′ side), for example, more towards the 5′ end than to the center of the minus-strand RNA virus genome (the position is further on the 5′ end than that of the gene in the middle). That is, among the multiple protein-coding sequences positioned on the genome, it is preferably positioned at a site closer to the 5′ end than the 3′ end (see the Examples). The MYC gene or (Hist gene can be positioned, for example, at the very end of the 5′ side (i.e., first position from the 5′ end), or at the second or third position from the 5′ end. The MYC gene or Glis1 gene may be positioned at the second position from the 5′ end of the genome, specifically, between the HN gene and the L gene (between HN-L) when the L gene is at the very end of the 5′ side of the genome with the HN gene next to it. In particular, the MYC gene or Glis1 gene is preferably inserted immediately upstream (3′ side, for example, between the HN gene and the L gene), or immediately downstream (5′ side, for example, between the HN gene and the 5′ trailer sequence) of the L gene in the Sendai virus genome. A Sendai virus vector in which the MYC gene has been inserted between the HN gene and L gene is most preferred. The Sendai virus vector may be, for example, an F gene-deleted Sendai virus vector (for example, the Z strain in which the M protein has G69E, T116A and A183S mutations; the HN protein has A262T, G264R, and K461G mutations; the P protein has L511F mutation; and the L protein has N1197S and K1795E mutations; and vectors produced by further introducing a TS 7, TS 12, TS 13, TS 14, or TS 15 mutation into this vector are more preferable.

MYC genes including not only wild type c-MYC but also the T58A mutant, N-MYC, and L-MYC can induce pluripotent stem cells (WO 2007/69666; Blelloch R. et al., Cell Stem Cell, 1:245-247, 2007). As such, since the family genes can be selected in various ways and used, reprogramming can be induced by appropriately selecting the MYC family genes. Furthermore, a continuous A or T nucleotide sequence of the MYC gene can be substituted by appropriately introducing silent mutations such that the encoded amino acid sequence is not changed.

For example, the amount of wild-type c-MYC expressed by an RNA virus vector such as a Sendai virus vector was found to be small. However, by introducing one or more, preferably two or more, three or more, or four or more mutations selected from a378g, t1122c, t1125c, a1191g, and a1194g, or all five of these mutations into the wild-type c-MYC, the gene can be highly expressed in astable manner from a vector. In the present invention, for example, the modified c-MYC gene shown in SEQ ID NO: 8 (SEQ ID NO: 9 for the amino acid sequence) (referred to as “c-rMYC”) can be used suitably. Specifically, examples include SeV(HNL)c-rMYC/TSΔF, SeV(L)c-rMYC/TSΔF, SeV(HNL)c-rMYC/TS15ΔF (WO 2010/008054; Fusaki et at., Proc. Jpn. Acad. Ser. B. Phys. Biol. Sci. Vol. 85, p348-362 (2009)), and preferably SeV(HNL)c-rMYC/TS15ΔF in particular, but are not limited thereto.

When appropriate, genes for cellular reprogramming and such can be loaded additionally into the above-mentioned Sendai virus vector carrying the KLF, OCT, and SOX genes, the Sendai virus vector containing the KLF gene but not the OCT gene and the SOX gene, and/or the Sendai virus vector containing the MYC gene. Furthermore, other vectors loaded with genes for cellular reprogramming and such may also be combined with the above-mentioned Sendai virus vectors. The genes to be loaded may be desired genes involved in the induction and such of various stem cells such as pluripotent stem cells from differentiated cells. For example, such genes that increase the efficiency of reprogramming can be loaded. The present invention provides uses of the Sendai virus vectors of the present invention for introducing genes in cellular reprogramming, and uses of these vectors for expressing reprogramming factors in cells to induce reprogramming of those cells. Furthermore, the present invention provides agents containing the Sendai virus vectors of the present invention for introducing genes in cellular reprogramming (transfer agents, gene transfer agents) and agents containing these vectors for expressing reprogramming factors in cells. Furthermore, the present invention relates to agents containing the Sendai virus vectors of the present invention for expressing reprogramming factors in cells to induce reprogramming of the cells. Furthermore, when carrying out nuclear reprogramming of cells, the vectors of the present invention are also useful for expressing desired genes in these cells. Sendai virus vectors of the present invention can be utilized for cellular reprogramming according to the present invention. The induction of reprogramming may be specifically an induction of pluripotent stem cells. The present invention can be used for medical uses and for non-medical uses, and is useful in medical and non-medical embodiments. For example, the present invention can be used for therapeutic, surgical, and/or diagnostic, or non-therapeutic, non-surgical, and/or non-diagnostic purposes. In the present invention, a nuclear reprogramming factor refers to a gene used, by itself or together with a number of factors, for inducing a differentiated state of a certain cell to change to a more undifferentiated state, or a product thereof, and includes for example, a gene used for inducing dedifferentiation of differentiated cells, or a product thereof. The nuclear reprogramming factors in the present invention include factors essential for nuclear reprogramming and accessorial factors (auxiliary factors) which increase the efficiency of nuclear reprogramming. In the present invention, desired genes to be used for nuclear reprogramming can be loaded into a vector. For example, genes to be used for the production of pluripotent stem cells can further be loaded. Specifically, as the nuclear reprogramming factors for induction of pluripotent stem cells, for example, genes that are expressed in ES cells or early embryo but are not expressed or whose expression is decreased in many differentiated somatic cells (ES cell-specific genes and such) can be used. Such genes are preferably genes that encode transcription factors, nucleoproteins, or such. Methods for identifying nuclear reprogramming factors are already known (WO 2005/80598), and in fact, genes identified using this method have been shown to be useful in reprogramming into pluripotent stem cells (WO 2007/69666).

The reprogrammed cells can be cultured in lymphocytic differentiation factors to generate B cells, or B cell progenitors, which can subsequently be converted to B regulatory cells.

For generation of B regulatory cells, the inventive method disclosed here comprises contacting one or more B-cells ex vivo with an isolated interleukin-35 (IL-35) protein, and culturing the one or more B-cells under conditions to provide one or more B-cells that produce IL-10. Interleukin-35 (IL-35) is a member of the IL-12 family of heterodimeric cytokines and is composed of Ebi3, a β chain subunit encoded by the Epstein-Barr virus (EBV)-induced gene 3 (also known as IL27b), and the IL12p35 α subunit encoded by IL-12α (see, e.g., Collison et al., Nature, 450:566-569 (2007); Hunter et al., Nature Reviews, 5: 521-531 (2005); Devergne et al., Proc. Natl. Acad. Sci. USA, 94: 12041-12046 (1997); and Niedbala et al., Eur. J. Immunol., 37, 3021-3029 (2007)). IL-35 is produced by regulatory T-cells (Treg) and is required for the immunosuppressive activities of Tregs (see, e.g., Collison et al., supra, and Chaturvedi et al., J. Immunol., 186: 6641-6646 (2011)).

In some embodiments IL-35 is injected into a patient suffering from disc degenerative disease in order to induce in vivo expansion of B regulatory cells. The isolated IL-35 protein can be a native IL-35 that is isolated from regulatory T-cells which naturally produce IL-35. In this embodiment, the IL-35 protein preferably is isolated from a mammal (e.g., a human or a mouse). Alternatively, the isolated IL-35 can be a recombinant IL-35 protein (rIL-35) generated using routine molecular biology techniques. A recombinant IL-35 protein can contain all or a portion of a native IL-35 protein isolated from a human or a mouse. For example, a recombinant IL-35 protein can contain an entire native human IL-35 protein or an entire native mouse IL-35 protein. In another embodiment, a recombinant IL-35 protein can contain a portion of a native IL-35 protein isolated from a human and a portion of a native IL-35 protein isolated from a mouse (i.e., a “chimeric” IL-35 protein). One of ordinary skill in the art will appreciate that a recombinant IL-35 protein can contain other elements that optimize the expression and/or stability of the IL-35 protein in B-cells. In a preferred embodiment, the isolated IL-35 protein is a recombinant fusion protein comprising an IL-12p35 α subunit protein and an Epstein-Barr virus (EBV)-induced gene 3 (Ebi3) protein. Nucleic acid sequences that encode an IL-12p35 α subunit include, for example SEQ ID NO: 1, and NCBI Accession No. NM_000882.3. Nucleic acid sequences that encode an Ebi3 protein include, for example, SEQ ID NO: 2, and NCBI Accession No. NM_005755.2. Amino acid sequences of the IL-12p35 α subunit and the Ebi3 protein also are known and publicly available (see, e.g., NCBI Accession Nos. NP_001152896.1 and NP_032377.1 (IL-12p35 a) and NCBI Accession Nos. ABK41923.1 and AAH08209.1 (Ebi3)).

The one or more B-cells are contacted ex vivo. “Ex vivo” refers to methods conducted within or on cells or tissue in an artificial environment outside an organism with minimum alteration of natural conditions. In contrast, the term “in vivo” refers to a method that is conducted within living organisms in their normal, intact state, while an “in vitro” method is conducted using components of an organism that have been isolated from its usual biological context.

The isolated IL-35 protein can be introduced into a cell, preferably a B-cell, using any suitable method known in the art. For example, a nucleic acid sequence encoding an IL-35 protein can be introduced into the cells by “transfection,” “transformation,” or “transduction.” The terms “transfection,” “transformation,” or “transduction,” as used herein, refer to the introduction of one or more exogenous polynucleotides into a host cell by physical or chemical methods. Many transfection techniques are known in the art and include, for example, calcium phosphate DNA co-precipitation (see, e.g., Methods in Molecular Biology, Vol. 7, E. J. Murray (ed.), Gene Transfer and Expression Protocols, Humana Press (1991)), DEAE-dextran, electroporation, cationic liposome-mediated transfection, tungsten particle-facilitated microparticle bombardment (see, e.g., Johnston, Nature, 346: 776-777 (1990)), and strontium phosphate DNA co-precipitation (see, e.g., Brash et al., Mol. Cell Biol., 7: 2031-2034 (1987)). Alternatively, the one or more B-cells can be cultured in a medium that contains suitable amounts of the isolated IL-35 protein. For example, the B-cell can be provided in a culture medium, and the IL-35 protein can be introduced into the culture medium per se or as a solution of the IL-35 protein in an appropriate solvent. The selection of suitable methods for transformation, culture, amplification, screening, and purification of B-cells are known in the art (see, e.g., Kumar et al., Immunol. Lett., 47(3): 193-197 (1995); Whitlock et al., Proc. Natl. Acad. Sci. USA, 79(11): 3608-3612 (1982); Whitlock et al., J. Immunol. Methods, 67(2): 353-369 (1984); and Janeway et al., supra).

The one or more B-cells preferably are obtained or derived from a mammal, more preferably a mouse, and most preferably a human. The one or more B-cells can be primary B-cells. The term “primary cell” refers to a cell that is isolated directly from living tissue (e.g., from a biopsy) and established for growth in vitro or ex vivo. In the context of the invention, primary B-cells can be isolated from any suitable source, including, for example, umbilical cord blood, peripheral blood, and spleen, and are available from a variety of commercial sources. Alternatively, the one or more B-cells are derived from a B-cell line or a cell line of pre-B lymphocyte origin. Such cell lines are available from the American Type Culture Collection (ATCC, Manassas, Va.) and include, for example, RAMOS cells (ATCC CRL-1596), Daudi cells (ATCC CCL-213), Jiyoye cells (ATCC CCL-87), MPC-11 cells (ATCC CCL-167), EB-3 cells (ATCC CCL-85), RPMI 8226 cells (ATCC CCL-155), Raji cells (CCL-86), and derivatives thereof.

For generation of B regulatory cells to be administered into the patient suffering from disc degeneration, the one or more B-cells are cultured under conditions so that one or more of the B-cells produce interleukin-10 (IL-10). IL-10 is a Type II cytokine and the founding member of a family of cytokines that include IL-19, IL-20, IL-22, IL-24, IL-26, IL-28, and IL-29 (Commins et al., J. Allergy Clin. Immunol., 121:1108-1111 (2008)). All of these cytokines bind to receptors with similar structures and activate the Janus kinase (JAK)/signal transducer and activator of transcription (STAT) signaling pathways. IL-10 exhibits the most potent anti-immune and anti-inflammatory activity of all the family members. The main biological function of IL-10 appears to be exerted on dendritic cells (DCs) and macrophages. IL-10 is a potent inhibitor of antigen presentation as well as the production of proinflammatory cytokines (see, e.g., Mosser and Zhang, Immunol. Rev., 226:205-218 (2008)). IL-10 is produced by several types of immune and non-immune cells, such as, for example, T-helper type 2 (Th2) cells, subsets of regulatory T-cells, CD8+ T-cells, human B-cells, monocytes, some subsets of dendritic cells, granulocytes, keratinocytes, epithelial cells, and tumor cells (Mosser and Zhang, supra).

In some embodiments, B regulatory cells are administered together with anti-inflammatory/immune modulating agents in order to increase therapeutic efficacy in treatment of disc degeneration. Therapeutic agents include gold salts, sulphasalazine, antimalarias, methotrexate, D-penicillamine, azathioprine, mycophenolic acid, cyclosporine A, tacrolimus, sirolimus, minocycline, leflunomide, and glucocorticoids), a calcineurin inhibitor (e.g., cyclosporin A or FK 506), a modulator of lymphocyte recirculation (e.g., FTY720 and FTY720 analogs), an mTOR inhibitor (e.g., rapamycin, 40-O-(2-hydroxyethyl)-rapamycin, CCI779, ABT578, AP23573, or TAFA-93), an ascomycin having immuno-suppressive properties (e.g., ABT-281, ASM981, etc.), corticosteroids, cyclophosphamide, azathioprene, methotrexate, leflunomide, mizoribine, mycophenolic acid, mycophenolate mofetil, 15-deoxyspergualine, or an immunosuppressive homologue, analogue or derivative thereof, immunosuppressive monoclonal antibodies (e.g., monoclonal antibodies to leukocyte receptors such as MHC, CD2, CD3, CD4, CD7, CD8, CD25, CD28, CD40. CD45, CD58, CD80, CD86, or their ligands), other immunomodulatory compounds, adhesion molecule inhibitors (e.g., LFA-1 antagonists, ICAM-1 or-3 antagonists, VCAM-4 antagonists, or VLA-4 antagonists), a chemotherapeutic agent (e.g., paclitaxel, gemcitabine, cisplatinum, doxorubicin, or 5-fluorouracil), anti-TNF agents (e.g. monoclonal antibodies to TNF such as infliximab, adalimumab, CDP870, or receptor constructs to TNF-RI or TNF-RII, such as ENBREL™ (Etanercept) or PEG-TNF-RI), blockers of proinflammatory cytokines, IL-1 blockers (e.g., KINERET™ (Anakinra) or IL-1 trap, AAL160, ACZ 885, and IL-6 blockers), chemokine blockers (e.g., inhibitors or activators of proteases), anti-IL-15 antibodies, anti-IL-6 antibodies, anti-CD20 antibodies, NSAIDs.

When the inventive method comprises administering to a patient suffering from disc degeneration an isolated IL-35 protein, the IL-35 protein is administered at a dose sufficient to induce the generation of B-cells that produce IL-10 and suppress disc degeneration in the mammal. A typical dose can be, for example, in the range of 0.001 to 1000 μg; however, doses below or above this exemplary range are within the scope of the invention. The daily parenteral dose can be about 0.1 μg/kg to about 100 mg/kg of total body weight (e.g., about 5 μg/kg, about 10 μg/kg, about 100 μg/kg, about 500 μg/kg, about 1 mg/kg, about 50 mg/kg, or a range defined by any two of the foregoing values), preferably from about 0.3 μg/kg to about 10 mg/kg of total body weight (e.g., about 0.5 μg/kg, about 1 μg/kg, about 50 μg/kg, about 150 μg/kg, about 300 μg/kg, about 750 μg/kg, about 1.5 mg/kg, about 5 mg/kg, or a range defined by any two of the foregoing values), more preferably from about 1 μg/kg to 1 mg/kg of total body weight (e.g., about 3 μg/kg, about 15 μg/kg, about 75 μg/kg, about 300 μg/kg, about 900 μg/kg, or a range defined by any two of the foregoing values), and even more preferably from about 0.5 to 10 mg/kg body weight per day (e.g., about 2 mg/kg, about 4 mg/kg, about 7 mg/kg, about 9 mg/kg, or a range defined by any two of the foregoing values). Therapeutic or prophylactic efficacy can be monitored by periodic assessment of treated patients. For repeated administrations over several days or longer, depending on the condition, the treatment is repeated until a desired suppression of disease symptoms occurs. However, other dosage regimens may be useful and are within the scope of the invention. The desired dosage can be delivered by a single bolus administration of the composition, by multiple bolus administrations of the composition, or by continuous infusion administration of the composition.

When the inventive method comprises administering to a mammal B-cells which have been engineered to produce IL-10, a composition comprising the IL-10-producing B-cells can be administered to a mammal using standard cell transfer and immunotherapy techniques in order to treat disc degeneration.

Examples of such techniques that may be applicable to disc degeneration i include autologous cell therapy, allogeneic cell therapy, and hematopoietic stem cell therapy. In a preferred embodiment, a composition comprising IL-10-producing B-cells is administered to a mammal via adoptive transfer methods (see, e.g, Riley et al., Immunity, 30: 656-665 (2009), and Janeway et al., supra). The IL-10-producing B-cells can be administered in any suitable amount, so long as the introduction of the IL-10-producing B-cells suppresses disc degeneration in the mammal. A typical amount of cells administered to a mammal (e.g., a human) can be, for example, in the range of one million to 100 million cells however, amounts below or above this exemplary range are within the scope of the invention. For example, the daily dose of IL-10-producing B-cells can be about 1 million to about 50 million cells (e.g., about 5 million cells, about 15 million cells, about 25 million cells, about 35 million cells, about 45 million cells, or a range defined by any two of the foregoing values), preferably about 10 million to about 100 million cells (e.g., about 20 million cells, about 30 million cells, about 40 million, about 60 million cells, about 70 million cells, about 80 million cells, about 90 million cells, or a range defined by any two of the foregoing values), more preferably about 10 million cells to about 50 million cells (e.g., about 12 million cells, about 25 million cells, about 35 million cells, about 45 million cells, or a range defined by any two of the foregoing values).

In some embodiments, B regulatory cells are administered together with accessory cells, or “primed” cells. In some embodiments B regulatory cells allow for the use of non-optimal cells such as senescent cells for disc regeneration. For example, when a population of primary chondrocytes are passaged about 3 or 4 times, their morphology typically changes to fibroblastic chondrocytes, in one embodiment B regulatory cells reduce the transformation of chondrocytes to fibroblastic chondrocytes. In another embodiment, the invention provides the use of B Regulatory cells to generate therapeutic effects with administration of fibroblastic chondrocytes. As primary chondrocytes are passaged, they begin to lose some of their chondrocytic characteristics and begin to take on the characteristics of fibroblastic chondrocytes. When these fibroblastic chondrocytes are incubated or “primed” with a cytokine such as a protein from the TGF-β superfamily, the cells regain their chondrocytic characteristics, which include production of collagen. This effect is amplified by contact with, or exposure to soluble factors produced by B regulatory cells. Such primed cells include fibroblastic chondrocytes, which have been incubated with TGFB1, and as a result have reverted to collagen producing chondrocytes. An advantage of using primed cells in retardation of intervertebral disc degeneration is the ease of creating useable chondrocytes for introduction into the intervertebral disc for production of collagen and otherwise maintenance of the cartilaginous matrix.

The cells may include without limitation primary cells or cells which have undergone about one to twenty passages. The cells may be connective tissue cells. The cells may include cells that have undergone a morphogenic change, wherein the priming causes reversion to the characteristics of the original cell. The cells may include without limitation chondrocytes, fibroblasts, or fibroblastic chondrocytes. Priming may occur by incubating the cells for a period of at least 40 hours, or from 1 to 40 hours, from 2 to 30 hours, from 3 to 25 hours, from 4 to 20 hours, from 5 to 20, from 6 to 18 hours, 7 to 17 hours, 8 to 15 hours, or 9 to 14 hours, with a cytokine, and then optionally separating the cytokine from the cells and injecting the primed cells into a cartilaginous defect site of interest in order to regenerate cartilage, preferably hyaline cartilage. In one aspect, the cytokine may be a member of the superfamily of TGF-β. In particular, the cytokine may be TGF-β, and in particular, TGF-β1.

The cytokine may be present in the priming incubation mix in an amount to sufficiently “prime” the chondrocyte to be useful in the intervertebral treatment method. In this aspect, the priming incubation mix may contain at least about 1 ng/ml of the cytokine. In particular, the mix may contain from about 1 to 1000 ng/ml, from about 1 to 750 ng/ml, from about 1 to 500 ng/ml, from about 1 to 400 ng/ml, from about 1 to 300 ng/ml, from about 1 to 250 ng/ml, from about 1 to 200 ng/ml, from about 1 to 150 ng/ml, from about 1 to 100 ng/ml, from about 1 to 75 ng/ml, from about 1 to 50 ng/ml, from about 10 to 500 ng/ml, from about 10 to 400 ng/ml, from about 10 to 300 ng/ml, from about 10 to 250 ng/ml, from about 10 to 200 ng/ml, from about 10 to 150 ng/ml, from about 10 to 100 ng/ml, from about 10 to 75 ng/ml, from about 10 to 50 ng/ml, from about 15 to 500 ng/ml, from about 15 to 400 ng/ml, from about 15 to 300 ng/ml, from about 15 to 250 ng/ml, from about 15 to 200 ng/ml, from about 15 to 150 ng/ml, from about 15 to 100 ng/ml, from about 15 to 75 ng/ml, from about 15 to 50 ng/ml, from about 20 to 500 ng/ml, from about 20 to 400 ng/ml, from about 20 to 300 ng/ml, from about 20 to 250 ng/ml, from about 20 to 200 ng/ml, from about 20 to 150 ng/ml, from about 20 to 100 ng/ml, from about 20 to 75 ng/ml, from about 20 to 50 ng/ml, from about 25 to 500 ng/ml, from about 25 to 400 ng/ml, from about 25 to 300 ng/ml, from about 25 to 250 ng/ml, from about 25 to 200 ng/ml, from about 25 to 150 ng/ml, from about 25 to 100 ng/ml, from about 25 to 75 ng/ml, from about 25 to 50 ng/ml, from about 30 to 500 ng/ml, from about 30 to 400 ng/ml, from about 30 to 300 ng/ml, from about 30 to 250 ng/ml, from about 30 to 200 ng/ml, from about 30 to 150 ng/ml, from about 30 to 100 ng/ml, from about 30 to 75 ng/ml, from about 30 to 50 ng/ml, from about 35 to 500 ng/ml, from about 35 to 400 ng/ml, from about 35 to 300 ng/ml, from about 35 to 250 ng/ml, from about 35 to 200 ng/ml, from about 35 to 150 ng/ml, from about 35 to 100 ng/ml, from about 35 to 75 ng/ml, from about 35 to 50 ng/ml, from about 40 to 500 ng/ml, from about 40 to 400 ng/ml, from about 40 to 300 ng/ml, from about 40 to 250 ng/ml, from about 40 to 200 ng/ml, from about 40 to 150 ng/ml, from about 40 to 100 ng/ml, from about 40 to 75 ng/ml, or from about 40 to 50 ng/ml. One method of practicing the invention may include incubating the cells with a cytokine and/or B regulatory cells for a certain length of time to create primed cells and optionally separating the cytokine from the cells, and injecting the primed cells into intervertebral disc or the site of interest near it. Alternatively, the cells may be incubated with the cytokine of interest for a time and the combination may be administered to the site of defect without separating out the cytokine.

It is to be understood that while it is possible that substances such as a scaffolding or a framework as well as various extraneous tissues may be implanted together in the primed cell therapy protocol of the present invention, it is also possible that such scaffolding or tissue not be included in the injection system of the invention. In a preferred embodiment, in the inventive somatic cell therapy, the invention is directed to a simple method of injecting a population of primed connective tissue cells to the intervertebral disc space. It will be understood by the artisan of ordinary skill that the source of cells for treating a human patient may be the patient's own cells, but that allogeneic cells as well as xenogeneic cells may also be used without regard to the histocompatibility of the cells. Alternatively, in one embodiment of the invention, allogeneic cells may be used having matching histocompatibility to the mammalian host. To describe in further detail, the histocompatibility of the donor and the patient are determined so that histocompatible cells are administered to the mammalian host. Also, juvenile chondrocytes may also be used allogeneically without necessarily determining the histocompatibility of the donor and the patient.

In one embodiment of the invention, because disc degenerative disease is a continuous process, the degenerating disc to which the cells are administered may be in any one of a number of degenerative states. In some discs, the degree of degeneration will require more or less B regulatory cells to be administered. For example, the degenerating disc may be an intact disc. The degenerating disc may be a herniated disc (i.e., wherein a portion of the annulus fibrosus has a bulge). The degenerating disc may be a ruptured disc (i.e., wherein the annulus fibrosus has ruptured and the bulk nucleus pulposus has exuded). The degenerating disc may be delaminated (i.e., wherein adjacent layers of the annulus fibrosus have separated). The degenerating disc may have fissures (i.e., wherein the annulus fibrosus has fine cracks or tears through which selected molecules from the nucleus pulposus can leak). In all of these degenerative states, the extra-cellular matrix of either the AF or NP is also degrading.

The present invention is directed to intra-operatively providing healthy, B regulatory cells into degenerated intervertebral disc of a patient. The cells may be delivered to either the nucleus pulposus or the annulus fibrosus or both for repair and restoration of each respective extra-cellular matrix. In other embodiments, said B regulatory cells may be administered in the peri-discal or peri-spinal areas. The inventors believe that B regulatory cells provide a special advantage for administration into a degenerating disc because they possess properties that will help them to more readily survive the relatively harsh environment present in the degenerating disc.

In one embodiment, the B regulatory cells are obtained from the patient's own bone marrow through treatment with interleukin-35. In other embodiments, adipose or muscle tissue may be the source of B regulatory cells. In a preferred embodiment B regulatory cells are allogeneic and derived from pluripotent stem cell sources. In some embodiments, the B regulatory ells to be administered to the disc are provided in a concentrated form. When provided in concentrated form, the cells can be uncultured. Uncultured, concentrated B regulatory cells can be readily obtained by centrifugation, filtration (selective retention), or immunoabsorption. If a matrix is administered together with the B regulatory cells, it has suitable mechanical properties, it can be used to restore the height of the disc space that was lost during the degradation process. The cells may be injected at the same time or concurrently with the matrix in the targeted area of the disc. When the cells are concentrated using the centrifugation process, they are deliverable to the disc in a pellet form in suspension. In another embodiment, the cells are delivered using a carrier. The carrier can comprise, or can be selected from, the group consisting of beads, microspheres, nanospheres, hydrogels, gels, polymers, ceramics, collagen and platelet gels.

The carrier, in solid or fluid form, can carry the cells in several different ways. The cells can be embedded, encapsulated, suspended or attached to the surface of the carrier. In one embodiment, the carrier encapsulates the cells, provides nutrients, and protects the cells when they are delivered inside the disc. After a period of time inside the disc, the carrier degrades and releases the cells. Specific types of the various carriers are described below. In some embodiments, the B regulatory cells are provided in a sustained release device (i.e., sustained delivery device). The administered formulation can comprise the sustained release device. The sustained release device is adapted to remain within the disc for a prolonged period and slowly release the mesenchymal stem cells contained therein to the surrounding environment. This mode of delivery allows the mesenchymal stem cells to remain in therapeutically effective amounts within the disc for a prolonged period. One or more additional therapeutic agents can also be delivered by a sustained delivery device. Synthetic scaffolds, such as fumaric-acid based scaffolds, have been designed and tailored to allow for attraction of certain cells and to provide direction for the cells to differentiate in desired areas. The cells can also be embedded in the scaffold and then injected into the target area without affecting the viability or proliferation of the cells. After implantation of the fumaric-acid based scaffold, it degrades over time and no further surgery is necessary to remove the scaffold.

Carriers can also comprise hydrogels. The cells are encapsulated in the polymer chains of the hydrogel after gelation. Hydrogels can be delivered in a minimally invasive manner, such as injection to the target area. The hydrogel is also resorbed by the body. Hydrogel properties such as degradation time, cell adhesion behavior and spatial accumulation of extracellular matrix can be altered through chemical and processing modifications. Hydrogels suitable for use in the present invention include water-containing gels, i.e., polymers characterized by hydrophilicity and insolubility in water. See, for instance, “Hydrogels”, pages 458-459, in Concise Encyclopedia of Polymer Science and Engineering, Eds. Mark et al., Wiley and Sons (1990), the disclosure of which is incorporated herein by reference in its entirety. Although their use is optional in the present invention, the inclusion of hydrogels can be highly advantageous since they tend to possess a number of desirable qualities. By virtue of their hydrophilic, water-containing nature, hydrogels can house viable cells, such as mesenchymal stem cells, and can assist with load bearing capabilities of the disc.

In one aspect the present invention discloses ex vivo and in vivo techniques for delivery of a DNA sequence of interest to the connective tissue cells of the mammalian host. The ex vivo technique involves culture of target mammalian cells, in vitro transfection of the DNA sequence, DNA vector or other delivery vehicle of interest into the mammalian cells, followed by transplantation of the modified mammalian cells to the target area of the mammalian host, so as to effect in vivo expression of the gene product of interest.

It is to be understood that while it is possible that substances such as a scaffolding or a framework as well as various extraneous tissues may be implanted together in the protocol of the present invention, it is preferred that such scaffolding or tissue not be included in the injection system of the invention. In a one embodiment, the invention is directed to a simple method of injecting a TGF superfamily protein or a population of cultured, untransfected/untransduced connective tissue cells or transfected/transduced mammalian cells or a mixture thereof to the intervertebral disc space so that the exogenous TGF superfamily protein is expressed or is active in the space.

It will be understood by the artisan of ordinary skill that one source of cells for treating a human patient is the patient's own cells. Another source of cells includes allogeneic cells without regard to the histocompatibility of the cells to the patient sought to be treated.

More specifically, this method includes employing a gene product that is a member of the transforming growth factor β superfamily, or a biologically active derivative or fragment thereof, or a biologically active derivative or fragment thereof.

In another embodiment of this invention, a compound for parenteral administration to a patient in a therapeutically effective amount is provided that contains a TGF-β superfamily protein and a suitable pharmaceutical carrier.

Another embodiment of this invention provides for a compound for parenteral administration to a patient in a prophylactically effective amount that includes a TGF-β superfamily protein and a suitable pharmaceutical carrier.

In therapeutic applications, the TGF-β protein may be formulated for localized administration. Techniques and formulations generally may be found in Remington's Pharmaceutical Sciences, Mack Publishing Co., Easton, Pa., latest edition. The active ingredient that is the TGF protein is generally combined with a carrier such as a diluent of excipient which may include fillers, extenders, binding, wetting agents, disintegrants, surface-active agents, erodable polymers or lubricants, depending on the nature of the mode of administration and dosage forms. Typical dosage forms include, powders, liquid preparations including suspensions, emulsions and solutions, granules, and capsules.

The TGF protein of the present invention may also be combined with a pharmaceutically acceptable carrier for administration to a subject. Examples of suitable pharmaceutical carriers are a variety of cationic lipids, including, but not limited to N-(1-2,3-dioleyloxy)propyl)-n,n,n-trimethylammonium chloride (DOTMA) and dioleoylphophotidyl ethanolamine (DOPE). Liposomes are also suitable carriers for the TGF protein molecules of the invention. Another suitable carrier is a slow-release gel or polymer comprising the TGF protein molecules.

The TGF beta protein may be mixed with an amount of a physiologically acceptable carrier or diluent, such as a saline solution or other suitable liquid. The TGF protein molecule may also be combined with other carrier means to protect the TGF protein and biologically active forms thereof from degradation until they reach their targets and/or facilitate movement of the TGF protein or biologically active form thereof across tissue barriers.

A further embodiment of this invention includes storing the cell prior to transferring the cells. It will be appreciated by those skilled in the art that the cells may be stored frozen in 10 percent DMSO in liquid nitrogen.

In the present application, a method is provided for regenerating or preventing degeneration of intervertebral disc by injecting an appropriate mammalian cell that is transfected or transduced with a gene encoding a member of the transforming growth factor-beta (TGF-β) superfamily, including, but not limited to, BMP-2 and TGF-β 1, 2, and 3.

In another embodiment of the present application, a method is provided for preventing or retarding degeneration of intervertebral disc by injecting an appropriate connective tissue cell that is not transfected or transduced with a gene encoding a member of the transforming growth factor-beta (TGF-β) superfamily or that is not transfected or transduced with any other gene. In another aspect, the invention is directed to treating injured or degenerated intervertebral disc by preventing or retarding degeneration of the intervertebral disc by using the above-described method.

In another embodiment of the present application, a method is provided for preventing or retarding degeneration of intervertebral disc by injecting an appropriate mammalian cell that is transfected or transduced with a gene encoding a member of the transforming growth factor-beta (TGF-β) superfamily. In another aspect, the invention is directed to treating injured or degenerated intervertebral disc by preventing or retarding degeneration of the intervertebral disc by using the above-described method.

In another embodiment of the invention, a method is provided for preventing or retarding degeneration of intervertebral disc by injecting a combination of or a mixture of an appropriate mammalian cell that is transfected or transduced with a gene encoding a member of the transforming growth factor-beta (TGF-β) superfamily and an appropriate connective tissue cell that is not transfected or transduced with a gene encoding a member of the transforming growth factor-beta (TGF-β) superfamily or that is not transfected or transduced with any other gene. In another aspect, the invention is directed to treating injured or degenerated intervertebral disc by preventing or retarding degeneration of the intervertebral disc by using the above-described method. In an embodiment of the invention, it is understood that the cells may be injected into the area in which degeneration of the intervertebral disc is to be sought to be prevented or retarded by using the cell above-described composition with or without scaffolding material or any other auxiliary material, such as extraneous cells or other biocompatible carriers. That is, the modified cells alone, unmodified cells alone, or a mixture or combination thereof may be injected into the area in which the degeneration of the intervertebral disc is sought to be prevented or retarded.

In one embodiment, the hydrogel is a fine, powdery synthetic hydrogel. Suitable hydrogels exhibit an optimal combination of properties such as compatibility with the matrix polymer of choice, and biocompatability. The hydrogel can include any one or more of the following: polysaccharides, proteins, polyphosphazenes, poly(oxyethylene)-poly(oxypropylene) block polymers, poly(oxyethylene)-poly(oxypropylene) block polymers of ethylene diamine, poly(acrylic acids), poly(methacrylic acids), copolymers of acrylic acid and methacrylic acid, poly(vinyl acetate), and sulfonated polymers. In general, these polymers are at least partially soluble in aqueous solutions, e.g., water, or aqueous alcohol solutions that have charged side groups, or a monovalent ionic salt thereof. There are many examples of polymers with acidic side groups that can be reacted with cations, e.g., poly(phosphazenes), poly(acrylic acids), and poly(methacrylic acids). Examples of acidic groups include carboxylic acid groups, sulfonic acid groups, and halogenated (preferably fluorinated) alcohol groups. Examples of polymers with basic side groups that can react with anions are poly(vinyl amines), poly(vinyl pyridine), and poly(vinyl imidazole). In accordance with the present invention, there is provided a method of treating degenerative disc disease in an intervertebral disc having a nucleus pulposus, comprising administering B regulatory cells into a degenerated intervertebral disc.

In one embodiment, B regulatory cells are generated from iPSC they are administered into the disc. In accordance with one aspect of the invention, the B regulatory cells can be delivered into the disc space with at least one (an) additional therapeutic agent, such as an agent to aid in the proliferation and differentiation of the cells. There can be, for example, one additional therapeutic agent (i.e., a second therapeutic agent) or there can be multiple additional therapeutic agents (e.g., second and third therapeutic agents). The additional therapeutic agent may be delivered simultaneously with the mesenchymal stem cells. In another embodiment, the additional therapeutic agent is delivered after administering the B regulatory to the disc. In yet another, the additional therapeutic agent is administered first, i.e., prior to administering the B regulatory cells to the disc.

The same carrier may also be used to deliver the cells and the additional therapeutic agent. In some embodiments, the cells are located on the surface of the carrier and the additional therapeutic agent is placed inside the carrier. In other embodiments, the cells and the additional therapeutic agent may be delivered using different carriers.

Other additional therapeutic agents which may be added to the disc include, but are not limited to: vitamins and other nutritional supplements; hormones; glycoproteins; fibronectin; peptides and proteins; carbohydrates (simple and/or complex); proteoglycans; oligonucleotides (sense and/or antisense DNA and/or RNA); bone morphogenetic proteins (BMPs); differentiation factors; antibodies (for example, antibodies to infectious agents, tumors, drugs or hormones); gene therapy reagents; and anti-cancer agents. Genetically altered cells and/or other cells may also be included in the matrix of this invention. If desired, substances such as pain killers (i.e., analgesics) and narcotics may also be admixed with the carrier for delivery and release to the disc space.

In some embodiments, growth factors are additional therapeutic agents. As used herein, the term “growth factor” encompasses any cellular product that modulates the growth or differentiation of other cells, particularly connective tissue progenitor cells. The growth factors that may be used in accordance with the present invention include, but are not limited to, members of the fibroblast growth factor family, including acidic and basic fibroblast growth factor (FGF-1 and FGF-2) and FGF-4, members of the platelet-derived growth factor (PDGF) family, including PDGF-AB, PDGF-BB and PDGF-AA; EGFs, members of the insulin-like growth factor (IGF) family, including IGF-I and-II; the TGF-β superfamily, including TGF-β1, 2 and 3 (including MP-52), osteoid-inducing factor (OIF), angiogenin(s), endothelins, hepatocyte growth factor and keratinocyte growth factor; members of the bone morphogenetic proteins (BMPs) BMP-1, BMP-3, BMP-2, OP-1, BMP-2A, BMP-2B, BMP-4, BMP-7 and BMP-14; HBGF-1 and HBGF-2; growth differentiation factors (GDFs), members of the hedgehog family of proteins, including indian, sonic and desert hedgehog; ADMP-1; GDF-5; and members of the colony-stimulating factor (CSF) family, including CSF-1, G-CSF, and GM-CSF; and isoforms thereof. The growth factor can be autologous such as those included in platelet rich plasma or obtained commercially. In one embodiment, the growth factor is administered in an amount effective to repair disc tissue.

In some embodiments, the growth factor is selected from the group consisting of TGF-β, bFGF, and IGF-1. These growth factors are believed to promote regeneration of the nucleus pulposus, or stimulate proliferation and/or differentiation of chondrocytes, as well as extracellular matrix secretion. In one embodiment, the growth factor is TGF-β. More preferably, TGF-β is administered in an amount of between about 10 ng/ml and about 5000 ng/ml, for example, between about 50 ng/ml and about 500 ng/ml, e.g., between about 100 ng/ml and about 300 ng/ml. In one embodiment, at least one of the additional therapeutic agents is TGF-B1. In one embodiment, another additional therapeutic agent is FGF. In some embodiments, platelet concentrate is provided as an additional therapeutic agent. In one embodiment, the growth factors released by the platelets are present in an amount at least two-fold (e.g., four-fold) greater than the amount found in the blood from which the platelets were taken. In some embodiments, the platelet concentrate is autologous. In some embodiments, the platelet concentrate is platelet rich plasma (PRP). PRP is advantageous because it contains growth factors that can restimulate the growth of the ECM, and because its fibrin matrix provides a suitable scaffold for new tissue growth.

In some embodiments, the cells may be introduced (i.e., administered) into the nucleus pulposus or the annulus fibrosus depending on which extra-cellular matrix needs rebuilding. In other embodiments, the cells may be introduced into both regions of the disc. Specific therapeutic agents may be selected depending on the region of the disc where the cells are going to be delivered. In embodiments, the cells alone are administered (e.g., injected) into the disc through a needle, such as a small bore needle. Alternatively, the formulation can also be injected into the disc using the same small bore needle. In some embodiments, the needle has a bore of about 22 gauge or less, so that the possibilities of producing a herniation are mitigated. For example, the needle can have a bore of about 24 gauge or less, so that the possibilities of producing a herniation are even further mitigated.

If the volume of the direct injection of the cells or formulation is sufficiently high so as to cause a concern of overpressurizing the nucleus pulposus, then it is preferred that at least a portion of the nucleus pulposus be removed prior to administration (i.e., direct injection) of the mesenchymal stem cells. In some embodiments, the volume of removed nucleus pulposus is substantially similar to the volume of the formulation to be injected. For example, the volume of removed nucleus pulposus can be within about 80-120% of the volume of the formulation to be injected. In addition, this procedure has the added benefit of at least partially removing some degenerated disc from the patient.

When injecting the B regulatory cells into the nucleus pulposus, it is desirable that the volume of drug (i.e., formulation of cells suspended in growth medium or a carrier) delivered be between about 0.5 ml and about 3.0 ml comprising cells suspended in growth medium or a carrier. When injected in these smaller quantities, it is believed that the added or replaced volume will not cause an appreciable pressure increase in the nucleus pulposus. Factors to consider when determining the volume of drug to be delivered include the size of the disc, the amount of disc removed and the concentration of the mesenchymal stem cells in the growth medium or carrier.

1. A method of preventing, reducing, or reversing disc degeneration comprising the steps of:
a) obtaining a population of B regulatory cells;
b) identifying a patient with disc degeneration; and
c) administering said regulatory B cells into said patient with said disc degeneration.

2. The method of claim 1, wherein said disc degeneration is characterized by dehydration of the nucleus pulposus.

3. The method of claim 1, wherein said disc degeneration is associated with reduced concentrations of tissue inhibitor of metalloprotease (TIMP) activity.

4. The method of claim 3, wherein said reduced TIMP activity is associated with lower expression of TIMP-1 protein as compared to a healthy age-matched control.

5. The method of claim 1, wherein said B regulatory cells are generated by culture of B cell progenitors with mesenchymal stem cells.

6. The method of claim 5, wherein said mesenchymal stem cells are cultured in the presence of a histone deacetylase inhibitor.

7. The method of claim 6, wherein said histone deacetylase inhibitor is valproic acid.

8. The method of claim 5, wherein said mesenchymal stem cells are cultured in the presence of a toll-like receptor agonist.

9. The method of claim 8, wherein said toll-like receptor agonist is an activator of toll-like receptor 2.

10. The method of claim 9, wherein said agonist of toll-like receptor 2 is Pam3CSK4.

11. The method of claim 1, wherein said disc degeneration is associated with increased inflammatory cell number in the intradiscal area.

12. The method of claim 11, wherein said inflammatory cells are neutrophils.

13. The method of claim 12, wherein said neutrophils have an increased propensity to produce MMP9 as compared to neutrophils obtained from a healthy age-matched control.

14. The method of claim 1, wherein an immune modulator is administered together with said perinatal tissue-derived exosomes to increase B regulatory cell numbers and/or activity.

15. The method of claim 14, wherein said B regulatory cells exhibit one or more of the following properties: express AIRE, GITR ligand, membrane-bound TGF-beta, Fas ligand, IL-10 receptor, IL-35 receptor, membrane-bound CTLA4, membrane-bound CD25, or TIM3; Inhibit T cell proliferation or block B cell proliferation; Inhibit Th1 cytokine production, including interferon gamma, alpha, beta, or interleukins 7, 12, 15, 18;
suppress generation of Th17 cells, Th9 cells, or NK cell activity; enhance generation of myeloid suppressor cells with capabilities such as differentiation into neutrophils or monocytes, expression of STAT3, or production of factors like PGE2, nitric oxide, TGF-beta, interleukin-10, interleukin-35, or leukemia inhibitory factor.

16. The method of claim 15, wherein said T cell proliferation is stimulated by one or more factors selected from the group consisting of: activation of the T cell receptor, TCR zeta chain, CD3, CD3 with a costimulatory molecule, including CD28, interleukin-7, interleukin-15, and mitogens, including lectins, and more specificially phytohemagglutinin and pokeweed mitogen.

17. The method of claim 14, wherein B regulatory cells are stimulated by administration of an agent selected from the group consisting of: anti-CD3 antibody, including low-dose IL-2, resveratrol, pterostilbene, quercetin, vitamin D3, ivermectin, n-acetylcysteine, and IVIG by itself or with low-dose IL-2 and mesenchymal stem cells.

18. The method of claim 17, wherein mesenchymal stem cells are derived from a tissue selected from the group consisting of: perivascular, omental, placental, testicular, endometrial, fallopian tube, bone marrow, peripheral blood, mobilized peripheral blood, deciduous teeth, menstrual blood, amniotic membrane, amniotic fluid, cerebral spinal fluid, trophoblastic tissue, dermal tissue, hair follicle, and lymphatic tissue.

19. The method of claim 18, wherein mesenchymal stem cells are pre-treated to increase homing to areas of inflammation via conditions or agents selected from the group consisting of: hypoxia, acidosis, hyperthermia, TNF-alpha, interferon gamma, and stimulators of indoleamine 2,3-dioxygenase.

20. The method of claim 14, wherein said B regulatory cells are generated from a pluripotent stem cell source selected from the group consisting of: somatic cell nuclear transfer-derived cells, parthenogenesis-derived stem cells, and induced pluripotent stem cells (iPSCs), wherein iPSCs are generated via transfection with one or more of OCT4, NANOG, Lin28, Sox-2, c-Myc, KLF4, Pim-1, or RAS.
